(19) United States
(12) Patent Application Publication
(10) Pub. No.: US 2006/0019331 A1
(43) Pub. Date: Jan. 26, 2006

(54) Method and system for generating a telephone alert indicating the presence of an analyte

(76) Inventor: Gideon Eden, Ann Arbor, MI (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/183,761

(22) Filed: Jul. 19, 2005

Related U.S. Application Data
(60) Provisional application No. 60/589,499, filed on Jul. 20, 2004.

(51) Int. Cl. C12Q 1/04; C12M 1/34

(52) U.S. Cl. 435/34; 435/287.1; 422/68.1

(57) ABSTRACT

A system is provided for detecting a presence or amount of an analyte in a sample. The system may include a detection device detecting a characteristic of a fluid in communication with the sample to generate an electrical signal corresponding to the characteristic of the fluid and a processor coupled to the detection device and determining at least one property relating to the analyte, based on the electrical signal. The system may also include a telephone interface device. The telephone interface device may be coupled to the processor and responsive to the processor to transmit an alarm message indicating the property of the analyte to at least one telephone communication device.

### CROSS REFERENCE

This application is based upon and claims the benefit of priority from U.S. Provisional Application No. 60/589,499 to Gideon Eden filed on Jul. 20, 2004, the entire contents of which are incorporated herein by reference.

### TECHNICAL FIELD

This disclosure relates generally to detecting an analyte and, more particularly, to detecting the presence or amount of an analyte in a sample.

### BACKGROUND

Microbial contamination may occur in various substances, such as food, pharmaceuticals, cosmetics, and water. Detection and analysis of such contamination may require estimations of total numbers of bacteria, yeasts, and molds, as well as concentrations of specific groups of organisms.

U.S. Pat. No. 5,366,873 issued on Nov. 22, 1994 to Eden et al. (the '873 patent) discloses a device for rapidly detecting microorganisms in a sample using semi-liquid material and electronic detection devices. Although this device is highly effective in many applications, it is desirable to efficiently communicate the detection of an alarm condition.

Methods and systems consistent with the present invention address one or more of the issues set forth above.

### SUMMARY

One aspect of the present invention includes a system for detecting a presence or amount of an analyte in a sample. The system may include a detection device detecting a characteristic of a fluid in communication with the sample to generate an electrical signal corresponding to the characteristic of the fluid and a processor coupled to the detection device and determining at least one property relating to the analyte, based on the electrical signal. The system may also include a telephone interface device. The telephone interface device may be coupled to the processor and responsive to the processor to transmit an alarm message indicating the property of the analyte to at least one telephone communication device.

Another aspect of the present invention includes a microbiological alert system for detecting contamination in a sample. The system may include a detection device detecting light from a fluid in communication with the sample to generate an electrical signal corresponding to the light from the fluid. The system may also include a processor coupled to the detection device and determining at least one parameter relating to a presence of microorganisms in the test sample based on the electrical signal and a telephone interface device. The telephone interface device may be coupled to the processor, and responsive to the processor to transmit an alarm message indicating the presence of microorganisms. Further, the presence of microorganisms may be determined. by the processor based upon the at least one parameter and a predetermined alarm criterion.

Another aspect of the present invention includes a microbiological alert system. The system may include detection means for generating data corresponding to at least one parameter relating to a property of an analyte in a test sample and processor means for receiving the data from the detection means. The system may also include means for storing processor instructions to be executed to determine an alarm state based upon at least the parameter and a specified alarm criterion and telephone interface means for transmitting an alarm message indicating the alarm state to an external telephone device under the control of the processor means.

Another aspect of the present invention includes a method for detecting and alerting a presence or amount of an analyte in a test sample. The method may include generating an electrical signal corresponding to a characteristic related to the analyte in the test sample and determining at least one parameter relating to the characteristic based on the electrical signal. The method may also include determining an alert condition indicating the presence of the analyte based upon the at least one parameter and a predetermined alarm criterion and communicating the alert condition to an external telephone device via a telephone communication channel.

It is to be understood that both the foregoing general description and the following detailed description are exemplary and explanatory only and are not restrictive of the invention, as claimed.

### BRIEF DESCRIPTION OF THE DRAWINGS

FIG. 1 is an illustration of an exemplary system for detecting the presence or amount of an analyte in a sample consistent with the present invention;

FIG. 2 illustrates a block diagram of a processor consistent with the present invention;

FIG. 3 illustrates a flowchart of an exemplary microbiological detection and alert process performed by a processor consistent with the present invention; and

FIG. 4 is an exemplary graph of microbiological detection.

### DETAILED DESCRIPTION

Reference will now be made in detail to the present embodiments of the invention, examples of which are illustrated in the accompanying drawings. Wherever possible, the same reference numbers will be used throughout the drawings to refer to the same or like parts.

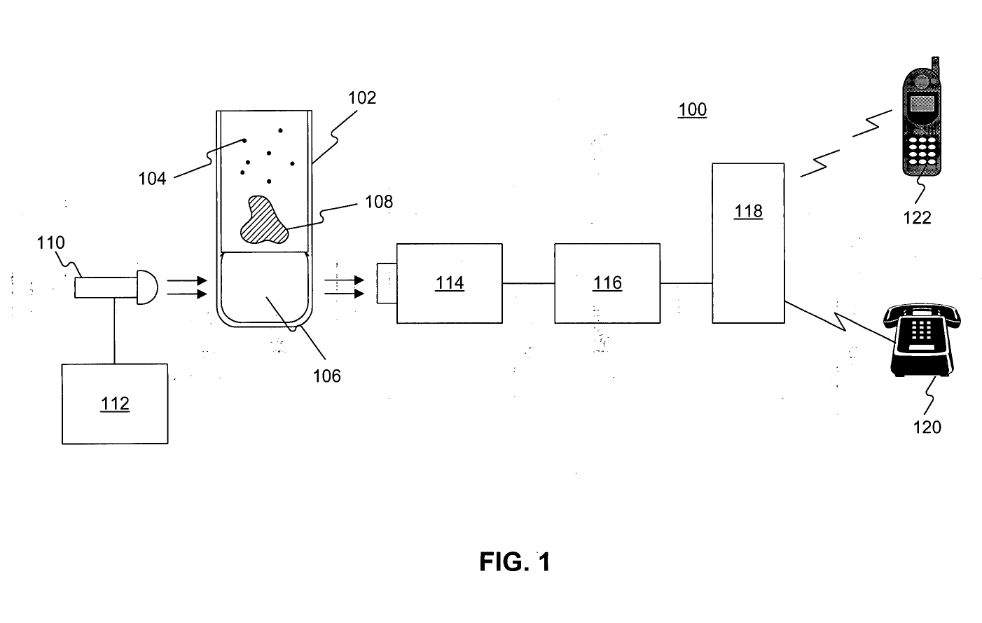

FIG. 1 shows an exemplary system 100 for detecting the presence of an analyte, for example, a containment. As shown in FIG. 1, system 100 may include a container such as a vial 102, a liquid media 104, a barrier layer, or region, 106, a test sample 108, a light source 110, and a light source controller 112. System 100 may also include a detection device 114, a processor 116, and a telephone interface device 118. Telephone interface device 118 may communicate with devices such as a land-line telephone 120 and/or a cellular telephone 122. It is understood that the devices and components listed are exemplary only, the number of the devices and components may be changed, and that other components may also be included.

Vial 102 may comprise any appropriate type of container made of transparent material (e.g., glass, transparent plastics, etc.) to hold test materials, such as liquid media 104, barrier region 106, test sample 108, etc. Liquid media 104 may comprise any appropriate type of fluid for cultivating microorganisms contained in test sample 108, such as plate count broth (Difco), etc. Liquid media 104 may also contain one or more indicator substances which are capable of undergoing certain types of transformation in the presence of microorganism growth. In some embodiments, the indicator substances may be capable of undergoing transformation in the presence of, or at a certain level of concentration of, an analyte, such as a microorganism, in test sample 108.

The transformation may be observed based on a certain characteristic of the fluid. For example, the color hue or intensity of the fluid may be changing. Alternatively, the way how light is reflected by the fluid and/or travels through the fluid may be changing. Other detectable characteristics may also be used.

Barrier region 106 may be disposed in vial 102 and may be a semi-fluid substance with a fluid portion and a non-fluid portion. The fluid portion of barrier region 106 may include a composition similar to or the same as liquid media 104, such that liquid media 104 and the fluid portion of barrier region 106 are in equilibrium. The non-fluid portion of barrier region 106 may include a gelling agent such as an appropriate type of agar (e.g., Muller Hinton Agar by Difco, Detroit, Mich.). Barrier region 106 may be positioned in vial 102 to facilitate measurement of changes in test sample 108.

Prior to introduction of test sample 108, both liquid media 104 and barrier region 106 may be sterilized, and liquid media 104 may be poured on the top of barrier region 106. After introduction of sample 108, microorganisms may be present in liquid media 106. However, both test sample 108 and microorganisms contained in test sample 108 are usually too large molecularly to penetrate the non-fluid portion of barrier region 106. Thus, barrier region 106 may be free of large molecules (e.g., test sample 108, microorganisms) and more suitable for providing accurate testing and detection results.

Initially, test sample 108, potentially containing microorganisms to be detected, may be placed in liquid media 104 of vial 102. Vial 102 may then be placed in an incubating device, at an appropriate temperature, to promote growth of any microorganisms of interest. The incubating device may include components such as an air incubator, heating and cooling blocks, or a heat exchanger. If microorganisms are indeed present in sample 108, the promoted growth of such microorganisms may result in changes in the composition of the liquid throughout vial 102, both the liquid in barrier region 106 and liquid media 104, because the liquid in barrier region 106 is in equilibrium with the remainder of the liquid in liquid media 104.

As explained above, the change in the composition of the liquid may be detected and measured in barrier region 106, which is in communication with test sample 108 but is free of test sample 108 and free of microorganisms. Thus, barrier region 106 provides a zone within which changes in the liquid, brought on by microorganism growth, can be readily detected and measured without any interference from the test sample.

As noted above, liquid media 104 may contain one or more indicator substances. In certain embodiments, the indicator substances may include a non-toxic indicator dye that changes in color hue or color intensity in the presence of microorganism growth. Examples of such indicator substances include pH indicators such as Bromcresol Purple, Phenol Red, Bromcresol Green, Bromphenol Blue, and Bromthymol Blue, and Redox indicators such as resazurin, methylene Blue, tetrazolium, and thionine. In operation, the indicator substance may be added to the liquid of both liquid media 104 and barrier region 106.

Further, the indicator substance may also include a luminescent substance that emits light as a result of microbial growth and metabolism such as ATP with luciferin/luciferase enzyme, or a chemiluminescent material such as luminal. Fluorescent materials, such as umbeliferons and coumarins may also be utilized.

The change in color hue or intensity of the indicator substances may be detected, monitored, and/or measured by measuring a parameter, such as light from light source 110 transmitted through barrier region 106. Light source 110 may be positioned at the bottom part of vial 102 such that light transmitted from light source 110 may be directed through transparent walls of vial 102 and through barrier region 106. Light source 110 may include any appropriate type of light source, such as incandescent lamps, gas-charged lamps, lasers, and light emitting diodes (LED's). In certain embodiments, a specific LED color, such as yellow, orange, green, and blue LED's, may be selected based upon spectral characteristics of barrier region 106. In applications using a luminescent material, light source 110 may not be required.

Light source 110 may be controlled by light source controller 112. Light source controller 112 may include any appropriate type of light source control system to provide electrical energy such that light from light source 110 is spatially and temporally uniform and stable.

Light transmitted from light source 110 may be detected by detection device 114 after passing through barrier region 106. The optical transmissive properties of barrier region 106 may be detected and monitored continuously and/or periodically. Additionally, other optical changes in barrier region 106, such as reflectance or fluorescence, may also be measured and analyzed. Further, multiple samples, each in a separate vial, may be used in system 100, with each vial 102 having a separate light source and detection device 114 to eliminate complex mechanical indexing devices utilized in optical readers. Other configurations, however, may also be used.

Detection device 114 may include any appropriate type of light detecting and signal processing device. Detection device 114 may include one or more light sensors (not shown) to convert dynamic changes in transmitted or reflected light, which may be indicators of bacterial activity, into electrical signals. The light sensors may include any appropriate types of sensors, such as photo voltaic sensors, photodiodes, phototransistors, photo multipliers, charged coupled devices (CCD), and/or multi-channel devices using low cost solid state sensors, etc.

Detection device 114 may also include other electronic components and/or devices, such as analog-to-digital (A/D) converters, to process electrical signals from the light sensors into corresponding output signals. The output signals may then be transmitted to processor 116 for further processing.

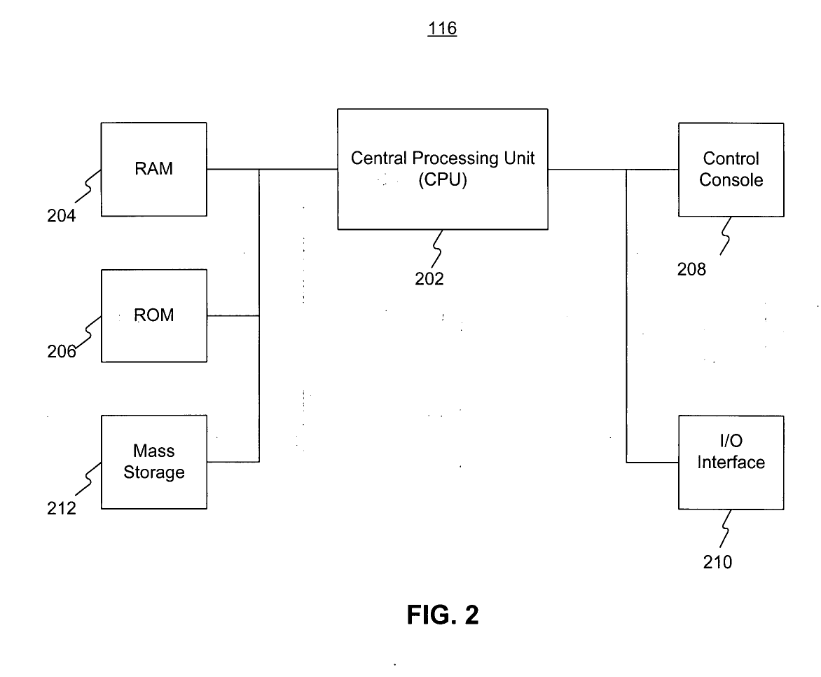

FIG. 2 shows an exemplary block diagram of processor116. As shown in FIG. 2, processor 116 may include a central processing unit (CPU) 202, a random access memory (RAM) 204, a read-only memory (ROM) 206, a control console 208, an I/O interface 210, and mass storage 212. It is understood that the type and number of listed devices are exemplary only and not intended to be limiting. The number of listed devices may be changed and other devices may be added.

CPU 202 may comprise any appropriate type of general purpose microprocessor, digital signal processor, or microcontroller, such as, for example, a Pentium IV. CPU 202 may execute sequences of computer program instructions to perform various processes. The computer program instructions may be loaded into RAM 204 for execution by CPU 202 from a read-only memory (ROM), or from mass storage 212. Mass storage 212 may include any appropriate type of mass storage device to store information that CPU 202 may need to perform the desired processes. For example, mass storage 212 may include one or more hard disk devices, optical disk devices, or other storage devices.

Control console 208 may provide a graphic user interface (GUI) to display information to operators and users of system 100. Control console 208 may include any appropriate type of computer display devices or computer monitors. Further, I/O interface 210 may be provided for CPU 202 to communicate with peripheral devices including detection device 114 and telephone interface 118.

Returning to FIG. 1, telephone interface 118 may be provided to permit system 100, especially processor 116, to communicate with various telephone devices, such as land-line telephone 120 and cellular telephone 122. Telephone interface 118 may include any appropriate type of telephone modem, such as a land-line telephone modem, a wireless/cellular telephone modem, a facsimile modem, etc.

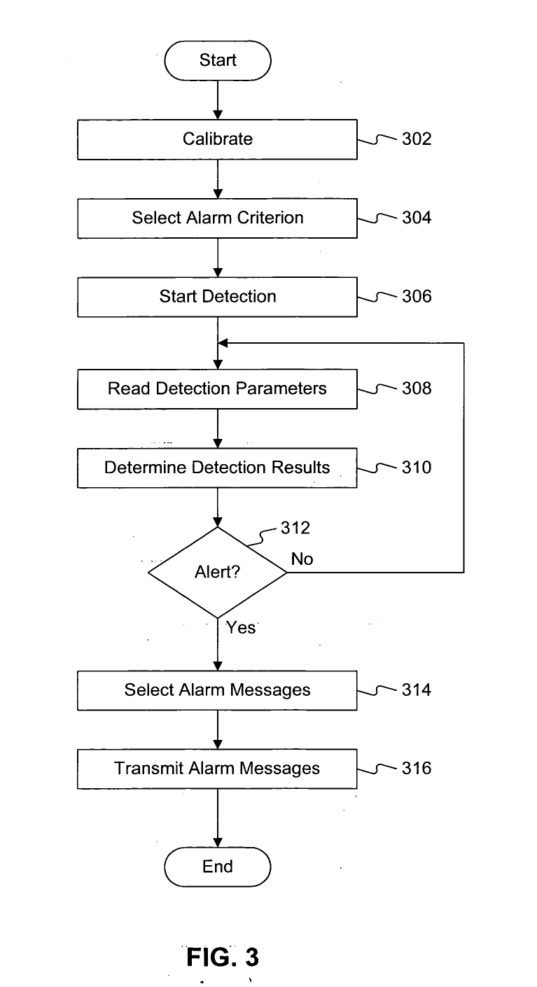

In operation, system 100 may perform a microbiological detection and alert process to accurately detect and report a property such as the presence and/or concentration level of an analyte, such as a microorganism, microorganism growth, and/or presence of a certain substance, etc. Multiple analytes, or substances, under analysis, may also be processed. FIG. 3 shows a flow chart diagram of an exemplary microbiological detection and alert process.

As shown in FIG. 3, at the beginning of the process, processor 116 may calibrate light source 110 and detection device 114 (step 302). Calibration may be performed without vial 102 to self-calibrate electronic components (e.g., light source 110, detection device 114, etc.). Other calibration methods may also be used.

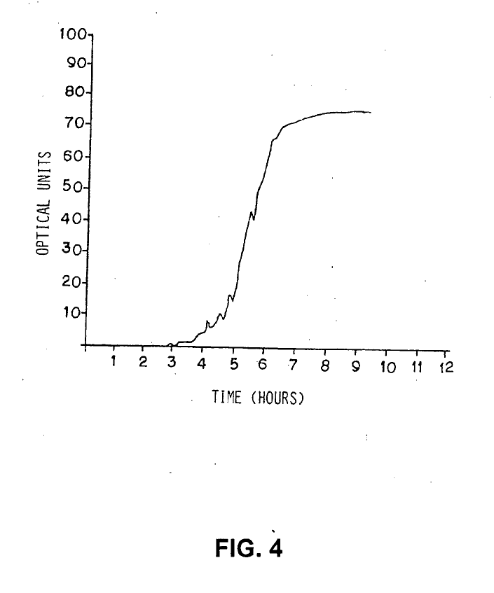

Returning to FIG. 3, after completing calibration, processor 116 may select an alarm criterion (step 304). The alarm criterion may be used to determine whether an alarm state has been reached. The alarm criterion may be selected based on a desired detection property of the microorganisms, such as contamination level, contamination time, etc. The alarm criterion may also be selected based on results of known test samples. For example, the selected alarm criterion may be an increase in growth rate occurring within a shorter time period than the rate indicated in FIG. 4. Other alarm criteria, however, may also be used.

Processor 116 may then start microbiological detection (step 306). As explained above, test sample 108 may be placed in vial 102 together with liquid media 104, barrier region 106, and indicator dye, such as Brom Crysol Purple, and incubated. The indicator dye may change its color from purple to yellow when the PH of liquid media 104 drops below 5.5. As liquid media 104 and barrier region 106 are in direct contact, small molecules (e.g., dye, H⁺, and/or other ions) may diffuse back and forth between liquid media 104 and barrier region 106, while barrier region 106 prevents microorganisms or other large molecules from entering. When vial 102 with test sample 108 is incubated, the microorganisms initially present in test sample 108 may grow and metabolize, thereby changing the PH of liquid media 104. At some point in time, the PH indicator may change color in liquid media 104, as well as in barrier region 106. The color change may then be detected by using light source 110 1ight source controller 112, and detection device 114.

After digital signals corresponding to the color change are obtained by processor 116 from detection device 114, processor 116 may determine optical readings or detection parameters based on the signals (step 308). Processor 116 may obtain optical readings at specific time intervals (e.g. 6 minutes apart) and store these readings in memory. Processor 116 may analyze these stored readings to determine the presence of an alarm condition. The alarm condition may refer to a property, such as a presence of a microorganism, a particular growth rate of the microorganism, and/or a concentration level of the microorganism in sample 108. The alarm condition may also refer to a presence or a concentration of a particular substance in test sample 108. When determining the alarm condition, processor 116 may record certain time parameters. For example, the time period after which a significant increase in rate of change of a detection parameter occurs may indicate the presence or absence of an alarm condition. This may be indicated by a detection time, that is, the point in time when substantial color change occurs.

Referring to FIG. 4, there is shown a graph of the output signal of detection device 114 vs. time. The units of the horizontal axis are hours and the units of the vertical axis are arbitrary units indicating the amount of light detected by the detection device 114, representative of color change. FIG. 4 indicates a detection time of about three hours, at which time a color change is detected. The detection time may be an indication of a presence of microorganisms in test sample 108 and a level of concentration of the microorganisms. Faster detection time may indicate a higher contamination of test sample 108. For example, a sample generating a 2 hour detection time may be more contaminated than a sample generating a 3 hour detection time.

Processor 116 may further determine a critical detection time (step 310). The critical time may be associated with a critical contamination level. In certain embodiments, processor 116 may predetermine a critical detection time or a critical contamination level. Processor 116 may also calculate the acceleration rate of microorganism growth or growth rate by analyzing readings in consecutive time intervals or time intervals in a certain sequence. The growth rate may be used to determine the presence and level of microorganisms in the original sample 108.

Further, processor 116 may determine whether an alert condition exists (step 312). If processor 116 determines a detection time faster than the predetermined critical detection time, or, alternatively, an acceleration rate larger than a predetermined growth rate, processor 116 may decide that an alert condition exists (step 312; YES). Otherwise, processor 116 may decide that an alert condition does not exist (step 312; NO). If there is no alert condition (step 312; NO), processor 116 may go back to step 308 to continue reading detection parameters.

On the other hand, if processor 116 decides that an alert condition exists (step 312; YES), processor 116 may generate an alarm message or messages to warn users of a detected contamination (step 314). Processor 116 may present the alarm message either audibly and/or visibly on control console 208. Processor 116 may also transmit an alarm message over telephone interface 118 to devices such as land-line telephone 120 and cellular telephone 122.

Processor 116 may connect via telephone interface 118 to a commercial telephone communication device to send an alarm message (step 316). For example, processor 116 may activate a dial-up telephone modem included in telephone interface 118 and dial a preprogrammed telephone number that receives the alert. Processor 116 may also generate a relevant voice message describing the nature of the alarm after dialing. Alternatively or in addition, processor 116 may generate alarm messages in the form of digital text messages that may be transmitted over specific telephone systems. For example, processor 116 may activate a wireless/cellular modem to use short message service (SMS) to transmit an alarm message to certain cellular telephones via an external SMS server in place of, or in addition to, transmitting a relevant voice message. In other embodiments, processor 116 may activate a facsimile modem to transmit an alarm message to one or more facsimile machines to provide a detailed printed alarm message. After sending out alarm messages, the microbiological detection and alert process may be completed.

Other embodiments of the invention will be apparent to those skilled in the art from consideration of the specification and practice of the invention disclosed herein.

What is claimed is:

1. A system for detecting a presence or amount of an analyte in a sample, comprising:
a detection device detecting a characteristic of a fluid in communication with the sample to generate an electrical signal corresponding to the characteristic of the fluid;
a processor coupled to the detection device and determining at least one property relating to the analyte, based on the electrical signal; and
a telephone interface device, coupled to the processor and responsive to the processor to transmit an alarm message indicating the property of the analyte to at least one telephone communication device.

2. The system of claim 1, wherein the characteristic comprises a light level.

3. The system of claim 1, wherein the property of the analyte comprises a concentration level of the analyte.

4. The system of claim 1, wherein the telephone interface device transmits the alarm message to a land-line telephone.

5. The system of claim 1, wherein the telephone interface device transmits the alarm message to a cellular telephone.

6. The system of claim 1, wherein the telephone interface device transmits the alarm message to a facsimile device.

7. A microbiological alert system for detecting contamination in a sample, comprising:
a detection device detecting a light from a fluid in communication with the sample to generate an electrical signal corresponding to the light from the fluid;
a processor coupled to the detection device and determining a presence of microorganisms in the test sample based on the electrical signal; and
a telephone interface device, coupled to the processor, and responsive to the processor to transmit an alarm message indicating the presence of microorganisms,
wherein the presence of microorganisms is determined by the processor based upon the at least one parameter and a predetermined alarm criterion.

8. The system of claim 7, wherein the telephone interface device comprises a telephone modem for transmitting the alarm message to one or more telephone devices.

9. The system of claim 7, further comprising:
a light source transmitting light to the fluid.

10. The system of claim 7, further comprising:
a liquid media containing the test sample, the liquid media facilitating growth of microorganisms and altering the value of at least one characteristic in response to metabolic byproducts of microorganisms.

11. The system of claim 10, wherein the processor comprises a processor analyzing the value of the at least one characteristic over time.

12. The system of claim 11, wherein the processor analyzes the value of the at least one characteristic in predetermined time intervals.

13. The system of claim 10, wherein the processor comprises a processor analyzing the value of the at least one characteristic over time, thereby determining at least one of the presence of or the level of microorganisms in the liquid media.

14. The system of claim 13, wherein the processor analyzes the value of the at least one characteristic in predetermined time intervals.

15. The system of claim 10, further comprising:
a barrier region in communication with the liquid media.

16. The system of claim 15, wherein the barrier region comprises a fluid portion and a non-fluid portion, the fluid portion being in equilibrium with the liquid media.

17. A microbiological alert system, comprising:
detection means for generating data corresponding to at least one parameter relating to a property of an analyte in a test sample;
processor means for receiving the data from the detection means;
means for storing processor instructions to be executed to determine an alarm state based upon at least the parameter and a specified alarm criterion; and
telephone interface means, controlled by the processor means, for transmitting an alarm message indicating the alarm state to an external telephone device.

18. The system of claim 17, wherein the property of the analyte includes a concentration level of a microorganism.

19. The system of claim 17, further comprising:
a liquid nutrient media facilitating growth of microorganisms and affecting the value of the parameter by detection of metabolic byproducts of the microorganism.

20. The system of claim 19, wherein the value of the parameter is analyzed over time to determine whether the metabolic byproducts are being generated.

21. The system of claim 17, wherein the telephone interface means comprises means for transmitting the alarm message to a land-line telephone.

22. The system of claim 17, wherein the telephone interface means comprises a telephone modem capable of dialing predetermined telephone numbers.

23. The system of claim 17, wherein the telephone interface means comprises means for transmitting a text message describing the alarm state.

24. The system of claim 17, wherein the telephone interface means comprises means for transmitting an alarm message in the form of a voice message describing the alarm state.

25. The system of claim 17, wherein the telephone interface means comprises means for transmitting the alarm message to a cellular telephone.

26. The system of claim 25, wherein the telephone interface means comprises means for communicating text messages utilizing an external SMS (Short Message Service) server.

27. The system of claim 17, wherein the telephone interface means comprises means for transmitting the alarm message to a facsimile device.

28. A method for detecting and alerting a presence or amount of an analyte in a test sample, comprising:
generating an electrical signal corresponding to a characteristic related to the analyte in the test sample;
determining at least one parameter relating to the characteristic based on the electrical signal;
determining an alert condition indicating the presence of the analyte based upon the at least one parameter and a predetermined alarm criterion; and
communicating the alert condition to an external telephone device via a telephone communication channel.

29. The method of claim 28, wherein the characteristic is light transmissibility, the method further comprising:
transmitting light to the test sample.

30. The method of claim 28, wherein the alert condition indicates level of microorganisms.

31. The method of claim 28, wherein communicating further comprises:
dialing a predetermined telephone number to connect to the external telephone device; and
sending an audible alarm message regarding the alarm state to the external telephone device.

32. The method of claim 31, further comprising:
sending a text alarm message to the external telephone device using short message service (SMS).
